(19) United States
(12) Patent Application Publication
(10) Pub. No.: US 2009/0053113 A1
(43) Pub. Date: Feb. 26, 2009

(54) Compact Horizontal Air Purifier

(76) Inventor: CuiMei Mai, GuangDong (CN)

Correspondence Address: DAVID AND RAYMOND PATENT FIRM, 108 N. YNEZ AVE., SUITE 128, MONTEREY PARK, CA, 91754, US

(21) Appl. No.: 11/886,693

(22) PCT Filed: Dec. 13, 2005
(86) PCT No.: PCT/CN2005/002163

§ 371 Date: Sep. 19, 2007

(30) Foreign Application Priority Data
Mar. 26, 2005 (CN) 200520056423.1

(51) Int. Cl. B03C 3/40; B03C 3/00; A61L 9/22

(52) U.S. Cl. 422/121; 422/186.12

(57) ABSTRACT

The present invention provides a compact horizontal air filter. The electrode array of the elements tending to be tainted with dust in the air filter is simple and convenient to generate clean and fresh air. The air filter comprises a case, a frame slidably inserted in and drew out of the case, a first array electrode fixed on the frame, a second array electrode fixed on the frame and being apart from the first array electrode, and a high voltage pulse generator. The electropositive particles in the air are attracted by the electronegative metal made collecting dust vane when they pass through a collecting dust chamber.

### BACKGROUND OF THE PRESENT INVENTION

1. Field of Invention

The present invention relates to a compact horizontal air purifier, and more particularly to a compact horizontal air purifier, which comprises an electrode set, having a hollow structure, horizontally supported in a casing in a detachably coupling manner, wherein the electrode set is electrified to carry negative ions for attracting particles in ambient air with position ions for removing dirt from ambient air in a fan-less manner.

2. Description of Related Arts

Using high-voltage pulse technology to generate ozone to purify air is well known and electrical power can directly transform to air flow without using mechanical driven devices as shown in FIG. 1. Generally speaking, this technology includes a first and a second arrays of electrodes spaced in a predetermined distance, wherein the first array electrodes are composed of a plurality of small annular electrodes, and the second array electrodes are composed of a plurality of large annular electrodes. A high-voltage pulse generator has a positive electrode and a negative electrode electrically connecting to small annular electrode and large annular electrode respectively, so as to generate an air flow from the first array electrodes to the second array electrodes. Most of the particles in the air flow can attracted to the surface of the first and second array electrodes by electro-static force, so as to purifying the air by eliminating the particles of dust, smoke or pollen. Furthermore, a certain amount of ozone is released between the first and second array electrodes to sterilize and disinfect the air.

Typically, the small annular electrode is made of string-shaped conductor and the large annular electrode is made of plate-shaped conductor with negative electricity. The ionized string-shaped conductor charges the particles in the air with positive charge which can be attracted by the plate-shape conductor charged negatively. Under this circumstance, up to 85-95% particles can be collected. When the concentration of ozone is high, many kinds of odor can be effectively eliminated. Also, the filter should not be often replaced, and the power consumption is low.

The large annular electrode usually is mounted on a detachable frame for being detached to be cleaned, and inserted back for use again. This method has an obvious flaw: the dirt is attracted not only to the second array electrode, but also to the first array electrode. If only the second array electrode is cleaned, the dirt on the first array electrode can obviously lower the efficiency of the air purifier, raise the power consumption.

However, there are two technical prejudices. First, the position of each small annular electrode of the first array should be placed on the center line of the two neighboring large annular electrodes of the second array, which not only increases the degree of accuracy, but also time and cost in manufacturing. Second, each small annular electrode is string-shaped which is more vulnerable and hard to clean. In other words, when the particles are attached, the purifying efficiency is significantly reduced and the dust-collecting chamber needs to be often cleaned. However, the procedure of cleaning is complicated and likely to damage the purifier. Noise can be generated easily when the dust-collecting board is not clean. Ozone, an irritating substance to human, can also be generated in this process.

To solve the problems stated above and satisfy the demand of consumers, an air purifier has to have the following features: operating without noise, high reduction efficiency in dirt, germ, and odors in the air, reducing ozone, lower power consumption and cost, and operating without changing the air filter. High Efficiency Particulate Air-filter or HEPA is light, attractive with germ and virus elimination ability such as ultraviolet. HEPA is able to deodorize and clean with increased pressure, and can be operated continuously for 24 hours a day and seven days a week to generate clean and fresh air.

The present invention provides an air purifying device with hollow metal dust-collecting board and a hollow metal rigid conductor, wherein the first and second array electrodes are easy to be detached, cleaned and manufactured, and not easy to break.

### SUMMARY OF THE PRESENT INVENTION

main object of the present invention is to provide a compact horizontal air purifier, wherein array electrodes are easy to attract dirt and particles, and easy to be mounted and detached, so as to produce clean and fresh air. For cleaning it, a string-shaped conductor can be pulled out by installing two hollow structures thereon to prevent the situation that the conductor is not freely moved in the purifier and the user has to spend too much effort to clean the purifier, and the dust or particles still remain in the purifier. The electropositive particles in the air are attracted by the electronegative metal made collecting dust vane when they pass through a collecting dust chamber thereof. The air purifier in the present invention is to efficiently reduce the germ, odor and particles in the room, provide a quiet operation and lower the energy consumption and manufacturing cost, such as HEPA which is light, attractive with germ and virus elimination ability and is able to deodorize and clean with increased pressure, and can be operated continuously for 24 hours a day and seven days a week to generate clean and fresh air.

Another object of the present invention is to provide a compact horizontal air purifier, wherein the first and second array electrodes are mounted in a frame that can be pulled out from the outer case for cleaning and can be inserted back afterwards to prevent labor-intensive installation.

Another object of the present invention is to provide a compact horizontal air purifier, comprising a negatively charged metal dust-collecting board with hollow collecting blades, which can be hollow square body, hollow triangular body, or hollow polygonal body.

Another object of the present invention is to provide a compact horizontal air purifier, the position of the small annular electrode and large annular electrode can be slightly deviated, such that the degree of accuracy in installation is reduced, and the efficiency is thus increased. Noise can be eliminated when the dust or particles are accumulated in the present invention.

Another object of the present invention is to provide a compact horizontal air purifier, wherein the small annular electrodes have same number and face to face with the large annular electrodes, but the number and position do not affect the performance of the air purifier in the present invention.

Another object of the present invention is to provide a compact horizontal air purifier, wherein the first and second array electrodes receive electrical power from the high-voltage pulse generator through the connectors on the frame, as the frame is inserted into a predetermined position of the outer case.

Another object of the present invention is to provide a compact horizontal air purifier, wherein the air purifier is made of stainless or other metals to prevent metal aging, and it is easy to maintain.

Another object of the present invention is to provide a compact horizontal air purifier, comprising two annular electrodes collecting and eliminating dust at the same time, and to prevent discharging primarily on the stainless plate.

Accordingly, in order to accomplish the above object, the present invention provides a compact horizontal air purifier comprising:

an outer case having a center opening and a plurality of parallelly and spacedly apart symmetric holes defining an air inlet portion and an air outlet portion;

a frame, which is slidably inserted into and pulled out through the center opening, having a top plate and a bottom plate;

a second array electrode comprising a parallelly and spacedly apart plate-shaped large annular electrodes mounted on the frame, wherein each of the large annular electrodes has one end mounted on the top plate and another opposed end mounted on one end of bottom plate, wherein each of the large annular electrodes has a hollow structure;

a first array electrode comprising a plurality of parallelly and spacedly apart plate-shaped small annular electrodes mounted at the frame to align with the large annular electrodes respectively, wherein each of the small annular electrode has one end mounted on the top plate and another opposed end mounted on the bottom plate, wherein the small annular electrodes are mounted at the air inlet portion, wherein a margin between each of the small annular electrodes and the corresponding large annular electrode is equal and is aligned face to face to avoid arc discharge, which results in dramatically leap of ozone, making user uncomfortable, wherein each of the small annular electrode has a hollow or solid structure; and

a high-voltage pulse generator having a positive electrode and a negative electrode electrically and selectively connected with the first array electrode and the second array electrode respectively as the frame is inserted into the predetermined position of the outer case, wherein the positive and negative electrodes of the high-voltage pulse generator are electrically coupled with the large and small annular electrodes respectively for minimizing an amount of ozone generated between the first and second array electrodes, wherein the positive and negative electrodes of the high-voltage pulse generator are electrically coupled with the small and large annular electrodes respectively for maximizing an amount of ozone generated between the first and second array electrodes, wherein at least one set of the small and large annular electrodes is needed to form a single structure, wherein the small annular electrodes are electrified to the large annular electrodes to produce electromotive force for forming a breeze of ozone air.

The large annular electrode can be formed as one or more of a hollow square body, hollow triangular body, or hollow polygonal body, wherein the number of small and large annular electrodes is equal or slightly different, and the small and large annular electrodes are facing with each other.

The first array electrode and second array electrode are mounted on the frame that can be pulled out from the case of the air purifier, so that the frame with the electrodes can be cleaned and inserted back to the outer case for use again.

The small annular electrodes and large annular electrodes are electrically connected with the small annular electrode connectors and the large annular electrode connectors on the bottom plate respectively, wherein the outer case comprises a base, which has a plurality of small annular electrode connectors having the same number with the small annular electrodes, and a plurality of large annular electrode connectors having the same number with the large annular electrodes. The small annular electrode connectors and the large annular electrode connectors are electrically connected with positive electrode and negative electrode of high-voltage pulse generator respectively. When the frame is inserted to the predetermined position of the outer case, the small and large annular electrode connectors on the bottom plate are electrically connected with the small annular electrode connectors and the large annular electrode connectors on the base respectively.

The small and large annular electrodes are made of well-conducted material, wherein the small annular electrode is mounted on the air inlet. The top plate of the frame comprises a handle bar thereon. The small and large annular electrodes form in pairs as a basic installation structure that the small and large annular electrodes are aligned to produce electromotive force for forming a breeze of ozone air.

The margin between each of the small annular electrode and the corresponding large annular electrode is the same and is align face to face to avoid arc discharge, which will result in dramatically leap of ozone, making user uncomfortable.

One or more group of annular structure comprising at least one small annular electrode and at least one large annular electrode is the basic structure. The small annular electrode is formed in hollow shape or in solid structure and the large annular is formed in hollow shape. The large and small annular electrodes are electrically connected with the positive electrode and negative electrode respectively for minimizing the amount of ozone being generated. The small and large annular electrodes are electrically connected with the positive electrode and negative electrode respectively for maximizing the amount of ozone being generated.

These and other objectives, features, and advantages of the present invention will become apparent from the following detailed description, the accompanying drawings, and the appended claims.

### BRIEF DESCRIPTION OF THE DRAWINGS

FIG. 1 is a sectional view of a conventional air purifier according to a preferred embodiment of the present invention.

FIG. 2 is an exploded perspective view of the frame and the first and second array electrodes according to the above preferred embodiment of the present invention.

FIG. 3 is a sectional view of A-A position in FIG. 2.

FIG. 4 is a schematic view of the large annular embodies as hollow square body, hollow triangular body, or hollow polygonal body according to the above preferred embodiment of the present invention.

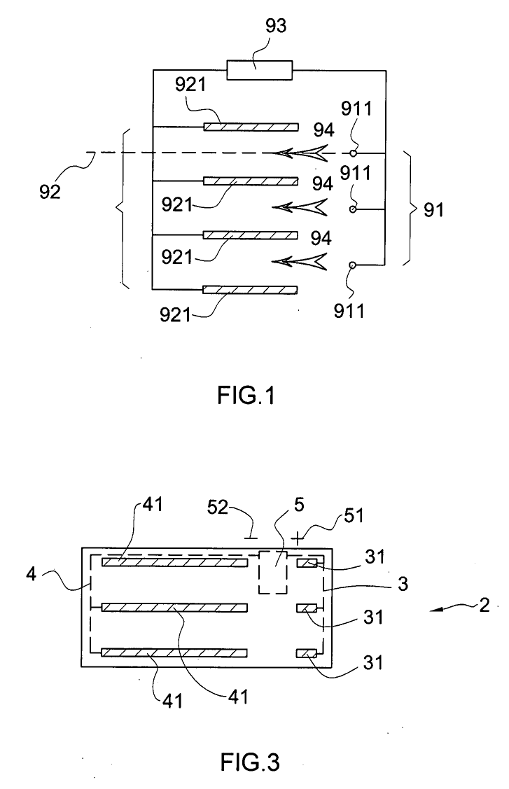

### DETAILED DESCRIPTION OF THE PREFERRED EMBODIMENT

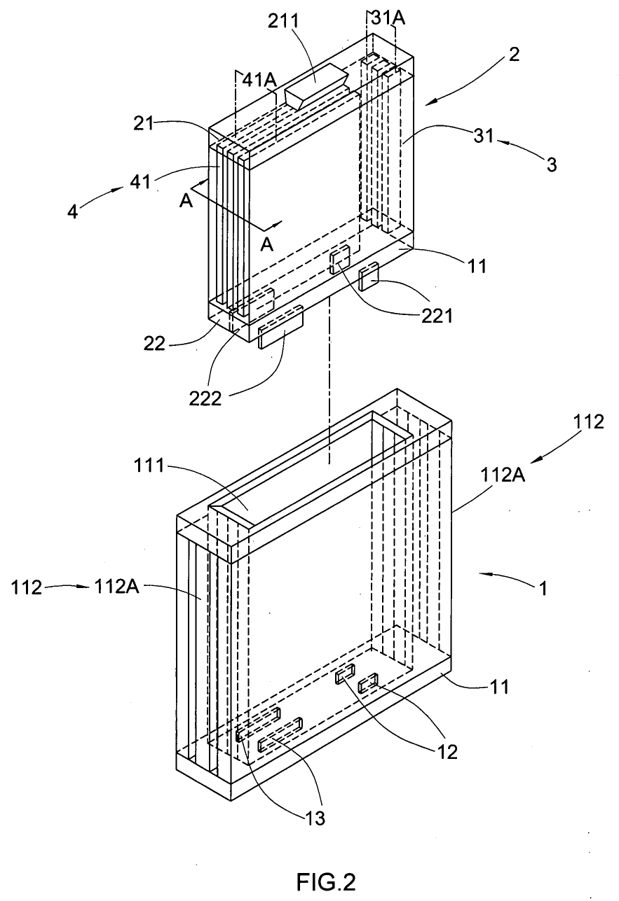

Referring to FIG. 2, an exploded perspective view of a compact horizontal air purifier according to a preferred embodiment of the present invention is illustrated, in which the compact horizontal air purifier comprises an outer case 1 having a center opening 111 and a plurality of parallelly and spacedly apart holes 112 at two sides of the outer case 1 to define a inlet port 112A and a outlet port 112B for an air flowing from one side of the outer case 1 to another opposed side thereof; a frame 2 which can be slidably inserted and pulled out from the center opening 111; a first array electrode 3 mounted on frame 2; a second array electrode 4 apart from the first array electrode 3; and a high-voltage pulse generator 5 having a positive electrode 51 and a negative electrode 52 electrically and selectively connected with the first array electrode 3 and the second array electrode 4 respectively as the frame 2 is inserted into the predetermined position of the outer case 1.

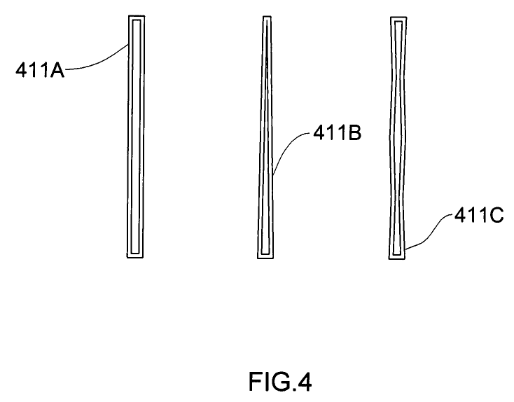

Referring to FIG. 3, a sectional view of FIG. 2, the first array electrode 3 comprises a plurality of parallelly and spacedly apart plate-shaped small annular electrodes 31. The second array electrode 4 comprises a plurality of parallelly and spacedly apart plate-shaped large annular electrodes 41 which are larger than the small annular electrodes 31. Accordingly, the small and large annular electrodes 31, 41 are the metal made hollow collecting dust vane. The frame 2 has a top plate 21 and a bottom plate 22, wherein one end of each of the large annular electrodes 41 and one end of each of the small annular electrodes 31 are mounted at the top plate 21 and an opposed end of each of the large annular electrodes 41 and an opposed end of each of the small annular electrodes 31 are mounted at the bottom plate 22. Each of the large annular electrodes 41 must be in hollow shape, wherein each of the small annular electrodes 31 can be in hollow shape or solid structure. The large annular electrodes 41 can be embodied as a hollow square body 411A, hollow triangular body 411B, or hollow polygonal body 411C, as shown in FIG. 4.

The first array electrode 3 mounted on the frame 2 comprises a plurality of parallelly and spacedly apart plate-shaped small annular electrodes 31, wherein one end of each of the small annular electrodes 31 is mounted on the top plate 21, and an opposed end of each of the small annular electrodes 31 is mounted on the bottom plate 22. The small annular electrode 31 is mounted at the air inlet. The margin between each of the small annular electrodes 31A and the corresponding large annular electrode 41A is the same, wherein the small annular electrodes 31 are aligned with the large annular electrodes 41A to avoid arc discharge, which will result in dramatically leap of ozone, making user uncomfortable. Each of the small annular electrodes 31A can be in hollow shape or in solid structure. In the preferred embodiment, the number of the small annular electrodes 31 equals to the number of the large annular electrodes 41 to align with each other, but the number of the small annular electrodes 31 and large annular electrodes 41 may vary and the position of them may be not perfectly aligned with each other, which may not affect the performance of the air purifier. Each small annular electrode 31 is electrically connected with a small annular electrode connector 221 on the bottom plate 22, and each large annular electrode 41 is electrically connected with a large annular electrode connector 222 on the bottom plate 22. Accordingly, the small and large annular electrode connectors 221, 222 are individual connectors spacedly formed at the bottom plate 22. Alternatively, the small and large annular electrode connectors 221, 222 are two common connectors spacedly formed at the bottom plate 22. The top plate 21 further comprises a handle bar 211 mounted thereon for inserting into and pulling out the frame 2 by the hand of the user. Both the large annular electrodes 41 and small annular electrodes 31 are plate-shaped conductor made of well-conducted materials with certain rigidity and hardness, such as stainless steel, nickel, carbon and so on.

The outer case 1 comprises a base 11 defining a plurality of small annular electrode contacts 221 for electrically coupling with the small annular electrode connectors 12 respectively and a plurality of large annular electrode contacts 222 for electrically coupling with the large annular electrode connectors 13 respectively when the frame 2 is slidably inserted into the outer case 1. The number of the small annular electrode contacts 221 equals to the number of the small annular electrode connectors 12. The number of the large annular electrode contacts 222 equals to the number of the large annular electrode connectors 13. The small annular electrode connector 12 and the large annular electrode connector 13 on the base 11 are electrically connected with the positive electrode and negative electrode of the high-voltage pulse generator 5 respectively. As the frame 2 is inserted into the predetermined position of the outer case 1, each small annular electrode contact 221 and large annular electrode contact 222 on the base 11 are electrically connected with the small annular electrode connector 12 and large annular electrode connector 13 to electrically connect with the small and large annular electrodes respectively.

The operation of the present invention is described explicitly hereinafter. For operating the air purifier, the frame 2 is slidably inserted into the predetermined position of the outer case 1. The small annular electrode connectors 221 and large annular electrode connectors 222 at the bottom plate 22 are electrically connected with the small annular electrode connectors 12 and large annular electrode connectors 13 at the base 11 respectively. The high-voltage pulse generator 5 outputs a series of high voltage pulse from 4 kV to 25 kV. The air between the first array electrode 3 and the second array electrode 4 is ionized to produce air flow and certain mount of ozone from the first array electrode 3 to the second array electrode 4. Most particles in the air flow are attracted by the electro static force on the small annular electrodes 31 and large annular electrodes 41 to purify the air. Certain amount of ozone can sterilize and disinfect the air. When the air purifier needs to be cleaned, the frame 2 is slidably pulled out with the first array electrode 3 and the second array electrode 4 mounted thereon, and the user can insert them back to the outer case 1 after cleaning.

In the present invention, the position of each small annular electrode 31 and large annular electrode 41 of the first and second array electrodes 3 and 4 of the compact horizontal air purifier can be alignedly face to face, or may have a little deviation, so that the degree of accuracy during manufacturing is reduced, and the production efficiency is thus increased. Furthermore, the small array electrodes 31 are high-strength plate-shaped instead of string-shaped, so that it can be easily cleaned and not easy to break. In addition, the first and second array electrodes 3 and 4 are mounted on the frame 2 that can be detachably pulled out from the outer case 1 of the air purifier to be cleaned and inserted back for use again, so that the small annular electrode 31 is easy to be cleaned, which improves the operation efficiency of the air purifier. Finally, when the frame 2 is inserted into the predetermined position of the outer case 1, the connectors 21 and 22 on the frame 2 are electrically connected with the contacts 12 and 13 on the base 11 of the outer case 1 respectively, so that the first and second array electrodes 3, 4 can get electrical power from the high-voltage pulse generator 5, which is very convenient.

The present invention provides a compact horizontal air purifier with the following installation requirements. The small annular electrode 31 must be smaller than its corresponding large annular electrode 41 wherein the smaller annular electrode 31 must be mounted at the air inlet. The margin of the small annular electrode 31 and its corresponding large annular electrode 41 must be equal and alignedly face to face to avoid arc discharge, which will result in dramatically leap of ozone, making people uncomfortable. The small annular electrode 31 can be formed in hollow shape or solid structure and its corresponding large annular electrode 41 must be in hollow shape. The small annular electrode 31 and large annular electrode 41 are electrically connected with the two electrodes of high-voltage pulse generator 5 respectively. If the large annular electrode 41 is connected with positive electrode and the small annular electrode 31 is connected with negative electrode, the release volume of the ozone is fewer. If the small annular electrode 31 is connected with positive electrode, and the large annular electrode 41 is connected with negative electrode, the release volume of the ozone is higher. At least one group of small annular electrode 31 and large annular electrode 41 are needed, and the small one discharges facing to the large one to produce electromotive force forming breeze. One or more groups of small annular electrode 31 and large annular electrode 41 are required to form an installation structure.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

1-23. (canceled)

24. A compact horizontal air purifier, comprising:
an outer case having a center opening and a plurality of spaced apart symmetric holes provided at two sides of said outer case to define an air inlet portion and an air outlet portion for allowing air flowing from one side of said outer case to an opposed side thereof;
a frame having a top plate and a bottom plate, wherein said frame is slidably inserted into said outer case through said center opening and is adapted to be pulled out from said outer case through said center opening;
a first array electrode comprising a plurality of spaced apart plate-shaped small annular electrodes extending in a parallel manner, wherein said small annular electrodes are mounted at said air inlet portion, wherein each of said small annular electrodes has one end mounted at said top plate and another opposed end mounted at said bottom plate;
a second array electrode comprising a plurality of spaced apart plate-shaped large annular electrodes extending in a parallel manner, wherein each of said large annular electrodes has a hollow structure, wherein each of said large annular electrodes has one end mounted at said top plate and another opposed end mounted at one end of bottom plate, wherein said large annular electrodes are aligned with said small annular electrodes, wherein a margin between each of said small annular electrodes and said corresponding large annular electrode is equal and is aligned face to face to avoid arc discharge, which results in dramatically leap of ozone, making user uncomfortable; and
a high-voltage pulse generator having a positive electrode and a negative electrode electrically and selectively connected with said first array electrode and said second array electrode respectively as said frame is inserted into a predetermined position of said outer case, wherein said positive and negative electrodes of said high-voltage pulse generator are electrically coupled with said large and small annular electrodes respectively for minimizing an amount of ozone generated between said first and second array electrodes, wherein said positive and negative electrodes of said high-voltage pulse generator are electrically coupled with said small and large annular electrodes respectively for maximizing an amount of ozone generated between said first and second array electrodes.

25. The compact horizontal air purifier, as recited in claim 24, wherein said first array electrode and said second array electrode are mounted at said frame, wherein when said frame is slidably pulled out from said outer case, said first array electrode and said second array electrode are detached from said outer case for cleaning, and when said frame is slidably pulled out from said outer case, said first array electrode and said second array electrode are slidably inserted back to said outer case for operation.

26. The compact horizontal air purifier, as recited in claim 24, wherein said bottom plate of said frame has a plurality of individual small annular electrode connectors electrically coupling with said small annular electrodes respectively for electrically coupling with said high-voltage pulse generator when said frame is slid into said outer case and a plurality of individual large annular electrode connectors electrically coupling with said large annular electrodes respectively for electrically coupling with said high-voltage pulse generator when said frame is slid into said outer case.

27. The compact horizontal air purifier, as recited in claim 25, wherein said bottom plate of said frame has a plurality of individual small annular electrode connectors electrically coupling with said small annular electrodes respectively for electrically coupling with said high-voltage pulse generator when said frame is slid into said outer case and a plurality of individual large annular electrode connectors electrically coupling with said large annular electrodes respectively for electrically coupling with said high-voltage pulse generator when said frame is slid into said outer case.

28. The compact horizontal air purifier, as recited in claim 26, wherein said outer case comprises a base defining a small annular electrode contact for electrically coupling with said small annular electrode connector and a large annular electrode contact for electrically coupling with said large annular electrode connector, wherein said small and large annular electrode connectors are electrically connected with said positive electrode and negative electrode of said high-voltage pulse generator respectively, wherein when said frame is inserted into said outer case, said small and large annular electrode connectors are electrically coupled with said small and large annular electrode connectors to electrically connect with said small and large annular electrodes respectively.

29. The compact horizontal air purifier, as recited in claim 27, wherein said outer case comprises a base defining a small annular electrode contact for electrically coupling with said small annular electrode connector and a large annular electrode contact for electrically coupling with said large annular electrode connector, wherein said small and large annular electrode connectors are electrically connected with said positive electrode and negative electrode of said high-voltage pulse generator respectively, wherein when said frame is inserted into said outer case, said small and large annular electrode connectors are electrically coupled with said small and large annular electrode connectors to electrically connect with said small and large annular electrodes respectively.

30. The compact horizontal air purifier, as recited in claim 24, wherein said bottom plate of said frame has a common small annular electrode connector electrically coupling with said small annular electrodes for electrically coupling with said high-voltage pulse generator when said frame is slid into said outer case and a common large annular electrode connector electrically coupling with said large annular electrodes for electrically coupling with said high-voltage pulse generator when said frame is slid into said outer case.

31. The compact horizontal air purifier, as recited in claim 25, wherein said bottom plate of said frame has a common small annular electrode connector electrically coupling with said small annular electrodes for electrically coupling with said high-voltage pulse generator when said frame is slid into said outer case and a common large annular electrode connector electrically coupling with said large annular electrodes for electrically coupling with said high-voltage pulse generator when said frame is slid into said outer case.

32. The compact horizontal air purifier, as recited in claim 30, wherein said outer case comprises a base defining a small annular electrode contact for electrically coupling with said small annular electrode connector and a large annular electrode contact for electrically coupling with said large annular electrode connector, wherein said small and large annular electrode connectors are electrically connected with said positive electrode and negative electrode of said high-voltage pulse generator respectively, wherein when said frame is inserted into said outer case, said small and large annular electrode connectors are electrically coupled with said small and large annular electrode connectors to electrically connect with said small and large annular electrodes respectively.

33. The compact horizontal air purifier, as recited in claim 31, wherein said outer case comprises a base defining a small annular electrode contact for electrically coupling with said small annular electrode connector and a large annular electrode contact for electrically coupling with said large annular electrode connector, wherein said small and large annular electrode connectors are electrically connected with said positive electrode and negative electrode of said high-voltage pulse generator respectively, wherein when said frame is inserted into said outer case, said small and large annular electrode connectors are electrically coupled with said small and large annular electrode connectors to electrically connect with said small and large annular electrodes respectively.

34. The compact horizontal air purifier, as recited in claim 25, wherein each of said large annular electrodes is formed in a hollow structure with one of hollow square body, hollow triangular body, and hollow polygonal body.

35. The compact horizontal air purifier, as recited in claim 29, wherein each of said large annular electrodes is formed in a hollow structure with one of hollow square body, hollow triangular body, and hollow polygonal body.

36. The compact horizontal air purifier, as recited in claim 33, wherein each of said large annular electrodes is formed in a hollow structure with one of hollow square body, hollow triangular body, and hollow polygonal body.

37. The compact horizontal air purifier, as recited in claim 25, wherein each of said small and large annular electrodes is a hollow collecting dust vane that a size of each of said small annular electrodes is smaller than a size of each of said large annular electrodes.

38. The compact horizontal air purifier, as recited in claim 35, wherein each of said small and large annular electrodes is a hollow collecting dust vane that a size of each of said small annular electrodes is smaller than a size of each of said large annular electrodes.

39. The compact horizontal air purifier, as recited in claim 36, wherein each of said small and large annular electrodes is a hollow collecting dust vane that a size of each of said small annular electrodes is smaller than a size of each of said large annular electrodes.

40. The compact air purifier, as recited in claim 38, wherein said frame further comprises a handle bar mounted to said top plate.

41. The compact air purifier, as recited in claim 39, wherein said frame further comprises a handle bar mounted to said top plate.

42. The compact air purifier, as recited in claim 40, wherein each of said small and large annular electrodes is made of stainless.

43. The compact air purifier, as recited in claim 41, wherein each of said small and large annular electrodes is made of stainless.
